(19) United States
(12) Patent Application Publication
(10) Pub. No.: US 2008/0262557 A1
(43) Pub. Date: Oct. 23, 2008

(54) OBESITY MANAGEMENT SYSTEM

(76) Inventor: Stephen J. Brown, Woodside, CA (US)

Correspondence Address: HEALTH HERO NETWORK, INC., 2400 GENG ROAD, SUITE 200, PALO ALTO, CA, 94303, US

(21) Appl. No.: 11/737,228

(22) Filed: Apr. 19, 2007

(51) Int. Cl. A61N 1/00; A61B 5/00; G08B 23/00

(52) U.S. Cl. 607/2; 340/693.1; 600/300

(57) ABSTRACT

A system, apparatus and method for obesity management through remote monitoring and feedback are described. In one embodiment of the present invention, periodic measurements of parameters affecting the obesity of a user are transmitted to a server through a user interactive communication device. Based on the information of the user, a customized feedback action is transmitted to the particular user. In another embodiment of the present invention an implantable apparatus having a neuro-stimulator device provides a therapeutic effect by stimulating a feeling of satiety to eating.

### BACKGROUND

A. Technical Field

This invention relates generally to remote health monitoring and feedback systems and more particularly to obesity management through remote monitoring.

B. Background of the Invention

The increased rate of obesity found in developed and developing societies around the world has become a serious health condition. The health hazards associated with obesity include increased deaths from diabetes, heart disease, stroke and certain types of cancer, and increased prevalence of osteoarthritis.

A future projection of current health trends clearly indicates a need to prevent obesity among children to safeguard them from the dangerous hazards associated with obesity. Lately, programs are increasingly being designed to assist a user in following a pre-designed schedule. Personalized and customized programs are also available.

However, the attention of a user forms an essence of such programs. Motivational programs are thus designed to increase the user attention to the programs. Further, to overcome the limitation of time and space, systems are designed to remotely monitor and assist users.

Expert opinion on training a user greatly depends on accuracy of measurements. Also, such measurements are observed to imply various effects for different users and thus require constant examination and personalization.

Food logging systems have also been designed to keep a record of user eating activity. However, such systems are found to be prone to errors that greatly affect the expert opinion. Further, neuro-stimulation techniques have been devised to control the desire of the user to eat. Such systems are learned to provide only a therapeutic affect on the user irrespective of diagnosis.

It is observed that understanding user activity and eating behavior provide important insights to designing a healthy eating and activity regime for the users. Therapeutic affect rightly combined with diagnosis of the user behavior needs to be devised to enable holistic management of the obesity epidemic.

Consequently, a simple to use and accurate system for measurement and feedback on details affecting obesity of the user is necessary.

### SUMMARY OF THE INVENTION

The present invention provides a system, apparatus and method for obesity management through remote monitoring and feedback. According to an embodiment herein provided, the system comprises of a server and a detecting device which is provided to be configured to intermittently measure various physical and eating activities of a user. Further, a user interactive communication device is provided, configured to intermittently receive information on the physical and eating activity from the detecting device. The user interactive communication device provided herein is configured to communicate with the server for sending information on physical and eating activity of the user, and receiving a feedback action.

In one embodiment of the present invention, periodic measurements of parameters affecting the obesity of a user are transmitted to a server through a user interactive communication device. The system is configured to be operable with a number of users.

In one embodiment of the present invention the detecting device in the system provides a therapeutic effect by providing a neuro-stimulator that stimulates a feeling of satiety to eating Therapeutic affect in addition to diagnosis provided by the intermittent measurement of various parameters provides a reliable system for obesity management of the user.

The present invention provides an accurate diagnosis by analyzing the user information accumulated in the server. A feedback action is designed by various expert applications, which may be linked with the server. The feedback action may be provided to the user with reports for better understanding of the progress.

Further, the present invention provides a method for continuous monitoring of the patient. The system of the present invention is so programmed that intermittent detection of the parameters occurs without user involvement thereby providing timely and detailed information. Use of holistic system for obesity management further ensures uniformity in various measurements. Also, the user interactive sessions for calibration ensure a much accurate feedback by the expert applications.

Other objects, features and advantages of the invention will be apparent from the drawings, and from the detailed description that follows below.

### BRIEF DESCRIPTION OF THE DRAWINGS

Reference will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. (“FIG.”) 1 is a general block diagram of the present invention as per one embodiment of the present invention.

FIG. 2 illustrates the system for obesity management according to one embodiment of the invention.

FIG. 3 illustrates an apparatus for intermittent detection of user parameters according to one embodiment of the invention.

FIG. 4 shows an apparatus for intermittent detection of user parameters according to another embodiment of the invention.

FIG. 5 shows a method for obesity management through remote monitoring and feedback as per one embodiment of the present invention.

### DETAILED DESCRIPTION OF THE PREFERRED EMBODIMENTS

A system, apparatus and method for obesity management through remote monitoring and feedback are described. In one embodiment of the present invention, periodic measurements of parameters affecting the obesity of a user are transmitted to a server through a user interactive communication device. Based on the information of the user, a customized feedback action is transmitted to the particular user. Objective measurement of physical and eating activity of a user are provided for timely measurement of the parameters.

In one embodiment of the present invention, a detecting device in the system provides a therapeutic effect by providing a neuro-stimulator that stimulates a feeling of satiety to eating; while objective measurement of the parameters provides a diagnostic input to a server for an overall obesity management of the user.

In the following description, for purpose of explanation, specific details are set forth in order to provide an understanding of the invention. It will be apparent, however, to one skilled in the art that the invention may be practiced without these details. One skilled in the art will recognize that embodiments of the present invention, some of which are described below, may be incorporated into a number of different remote patient monitoring systems. Structures and devices shown below in block diagram are illustrative of exemplary embodiments of the invention and are meant to avoid obscuring the invention. Furthermore, connections between components within the figures are not intended to be limited to direct connections. Rather, data between these components may be modified, re-formatted or otherwise changed by intermediary components.

Reference in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment.

A. Overview

FIG. 1 illustrates a general block diagram of an obesity management system 100 according to one embodiment of the invention. The system may cater to number of users at a given time.

A detecting device 103 may be placed with a user 101. The detecting device intermittently detects various physical and eating activity of the user. In one embodiment of the present invention the detecting device may be implanted within the user. The implantable feature of the embodiment allows placement of the detecting device within the body. The placement within the body may not be restricted to a particular organ, nerve or location, but allows a general placement within the body. The period at which the detection of the parameters by the detecting device occurs may be varied and programmed. The measurements include various parameters, which may affect and determine the obesity level of the user. The physical movement of the user is measured in order to examine the activity causing burning of calories in the body. Such information may provide useful inputs for determining user behavior and thereby designing a personalized program for the user. Further, details relating to eating activity may be detected intermittently by the detecting device 103.

Such information may be communicated from a detecting device 103 to a user interactive communication device 105. A remotely programmable device configured to receive information from the detecting device 103 and provide a user interface may be used as the user interactive communication device. The user interactive communication device may be a personal digital assistance device, which is configured to receive information from the detecting device. Alternatively, a personal computing device, for e.g. a personal computer, so configured may be used to receive information from the detecting device.

The information of the user available at the user interactive communication device 105 may be communicated to a server 107. The periodically obtained information may be maintained in the server 107, thereby keeping a track of the user details over a period of time. Such information available at the server and the instantaneous information available from the user interactive communication device may be used to device a feedback to the user.

In one embodiment of the present invention, an expert application 109 may be linked with the server 107. The feedback is devised by the expert application. The expert application may be pre-programmed such that a customized feedback action based on the user details may be automatically provided to the user.

The server 107 is configured to receive the feedback action from the expert application 109. The feedback action may be reformatted at the server 107 in order to provide user understandable details. Such feedback action may then be communicated to the user interactive communication device 105. The user interactive communication device 105 is configured to receive the feedback action and provide such information inform of a script or other user readable form.

The detecting device 103 may not require user involvement in providing the measurements at periodic intervals thereby leading to much detailed information available at the server. Convenience is also thus provided to the patients by relieving the patients from requiring periodic attention in providing their measurements. A feedback action based on the detailed user information may thus be more accurate.

B. Remote Expert Monitoring and Feedback

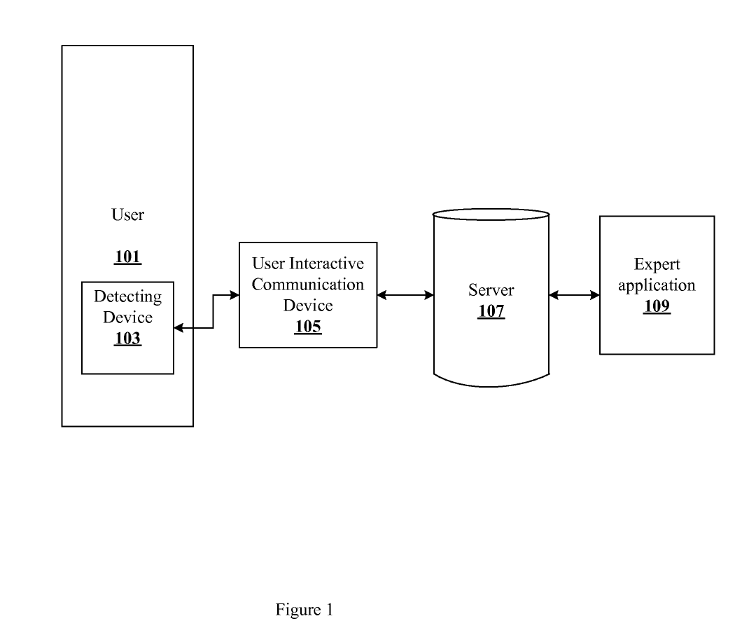

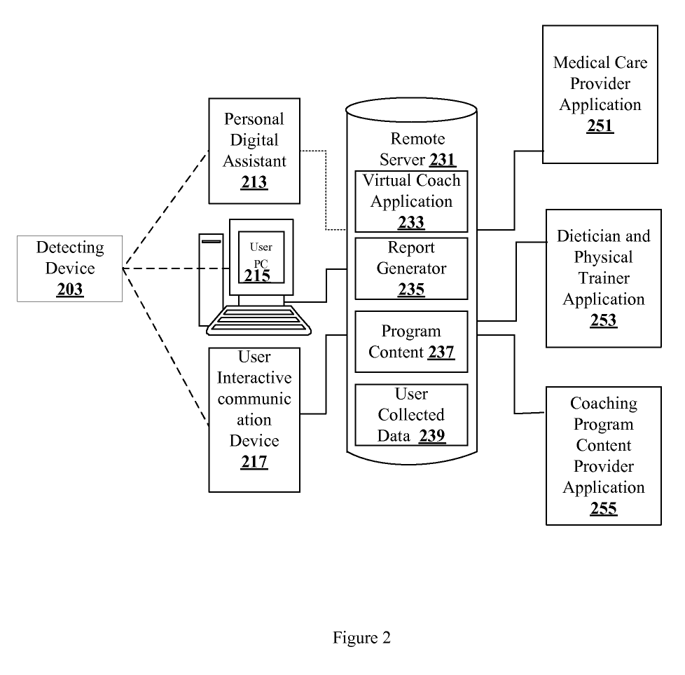

Referring to FIG. 2, various user interactive communication devices are used in the system. A personal digital assistance device 213 may be configured to receive the information from the detecting device. The detecting device may be wearable by the user. Alternatively, the detecting device may be implanted in the user. The communication from the detecting device to the user interactive communication device may be accomplished by providing a short-range communication device in the detecting device.

A personal computer 215 may communicate with the server 231 through an Internet connection. The personal digital assistance device 213 may communicate with the server through a wireless connection. Further, various remotely programmable devices configured to receive information from the detecting device may be used as the user interactive communication device. A wired communication of such device may be configured with the server for transmission and reception of to provide the information.

The server 231, which may be provided in the remote location, may comprise various applications. These applications may help in analyzing and reformatting the information obtained from the user. Particularly, a virtual coach application 233 may be available in the server 231. The virtual coach application may be programmed to respond to pre-decided set of situations. For e.g. an eating behavior, an adverse affect to the user may be programmed in the virtual coach application. Automated feedback action may be generated in response to such activity performed by the user. This allows the system to cater to an increased number of users by still providing a customized feedback.

Further expert applications may be linked to the server to provide specialized feedback action based on the user information. Such expert applications may also be made accessible to the users through the World Wide Web. More particularly, a medical care provider application 251 may be linked to the server 231. Feedback action on providing medical care if required may be decided by the medical care provider application 251. Further, a dietician and trainer application 253 may be linked to the server 231. Information obtained on food activity and physical activity of the user may be analyzed the dietician and physical trainer application to suggest an improved eating program and also device an activity schedule for the user.

Furthermore, a coaching program content provider application 255 may be linked with the server to provide a coaching schedule, which a user may perform. The feedback action devised by these expert applications may be provided to the server 231 where it may be reformatted by various server applications. A report generator 235 may be provided in the server to list the user information gathered. A report required for over-a-period information on the user may be provided to monitor the progress of the user.

Accumulation of data of the user may occur in the user collected data 239 which may be used by various expert applications. Graphical representations may be built on the basis of user collected data 239 for quicker analysis of the progress of the user. Such graphical representations may be used by any of the expert applications to provide feedback action. Also, such graphical representations may be sent to the user at the user interactive communication device 217 through any of the mediums discussed above.

C. Intermittent Objective Measurement

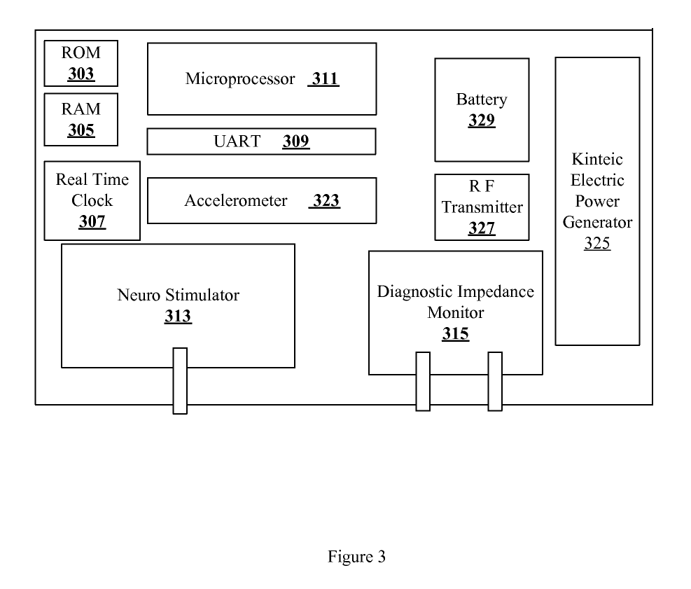

FIG. 3 illustrates an apparatus for physical and eating activity detection (detection device 103). The apparatus is configured to intermittently detect various parameters that affect the obesity levels of the user. In one embodiment of the present invention, the apparatus may be implanted in the user. Alternatively, in another embodiment of the present invention the apparatus may be wearable by the user.

The apparatus may comprise of a microprocessor 311, which controls various activities in the apparatus. Further, movement of data within the apparatus and transmission of measured parameters may be controlled by the microprocessor 311. A read only memory 303 may be used to store various instructions utilized by the microprocessor 311 and various other data.

A random access memory 305 may be used to store the measured values of the user. Synchronization of data transmission within the apparatus may be guided by a real time clock 307. The real time clock signal may also be used as a reference in transmission of data from the apparatus. A Universal Asynchronous Receiver Transmitter (UART) 309 may be used to perform the parallel-to-serial conversion of digital data to be transmitted and the serial-to-parallel conversion of digital data that has been transmitted.

The detection of physical activity of the user may be accomplished by an accelerometer 323, which detects physical movement of the user. A rechargeable power storage device in form of a battery 329 may be used for power storage for providing power to the apparatus. A power source may be provided in the apparatus for charging the battery 329. The power source may be provided in form of a kinetic electric power generator 325, which converts kinetic energy to electric current that may be used to charge the battery 329.

A neuro-stimulator 313 may be used in the apparatus to provide stimulations that effect appetite of the user. More particularly, the neuro-stimulator may generate pulses, which stimulate a feeling of satiety to eating. This feature of the invention provides a therapeutic affect to the user.

In order to measure the eating activity of the user a diagnostic impedance monitor 315 may be used. The electrical variations around the stomach may be measured to detect whether a user has consumed food. The diagnostic impedance monitor 315 may also measure emptying of the stomach of the user.

Calibration of the measurements provided by the diagnostic impedance monitor 315 may be done by having user interactive sessions. This ascertains the accuracy of such measurements. The calibration for each user may be based on the exclusive session of the user. Calibration may include translating the information on eating activity of the user obtained by measuring impedance around the stomach and muscle contraction into actual objective information about eating patterns. This enables an accurate and objective food logging for the specific user. Calibration of physical activity may be undertaken by translating the motion and vibrations sensed in the accelerometer into actual information about calories burned.

The calibration also ensures reliability of the measurements while providing an implantable detecting device. A general placement of the implanted detecting device may be allowed by accordingly calibrating the measurements by having user interactive sessions.

The information on eating activity of the user may further be analyzed along with the physical activity measurement to obtain a comparative study between food consumed and calories burned. Such study may be helpful in suggesting the user on changing his/her activity levels and/or changing their eating activity to meet a specified goal.

The detected parameters may be processed by the microprocessor to be transmitted to an external device. The transmission may occur by a radio-frequency transmitter 327. The RF transmitter may be configured to transmit information to a certain range based on the external device utilized to receive the transmission.

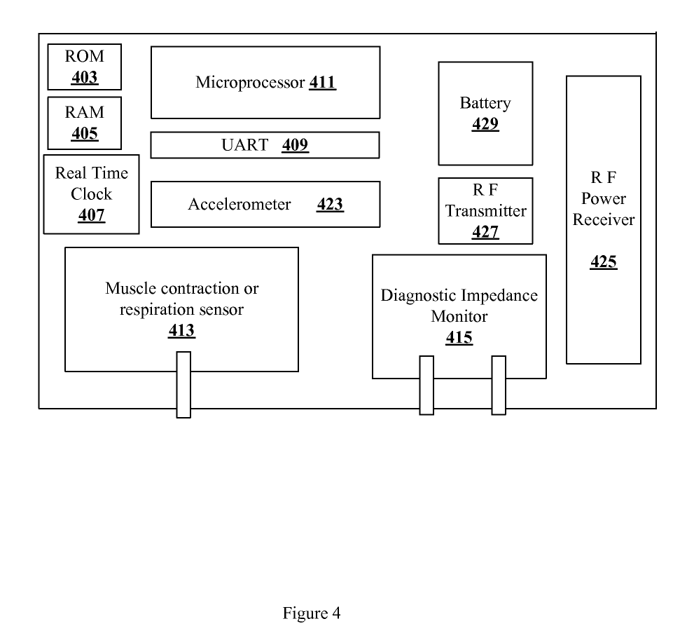

FIG. 4 shows another embodiment of the apparatus for physical and eating activity detection. In this embodiment a radio frequency power receiver (RF power receiver) 425 may be used to charge the battery 429. The power at the RF power receiver 425 may be received by the radio frequency signals, which may then be converted into electrical current that charges the battery 429.

Further, a muscle contraction or respiration sensor 413 may also be used with the diagnostic impedance monitor 415 to provide much accurate information on user eating activity.

D. Holistic Obesity Management

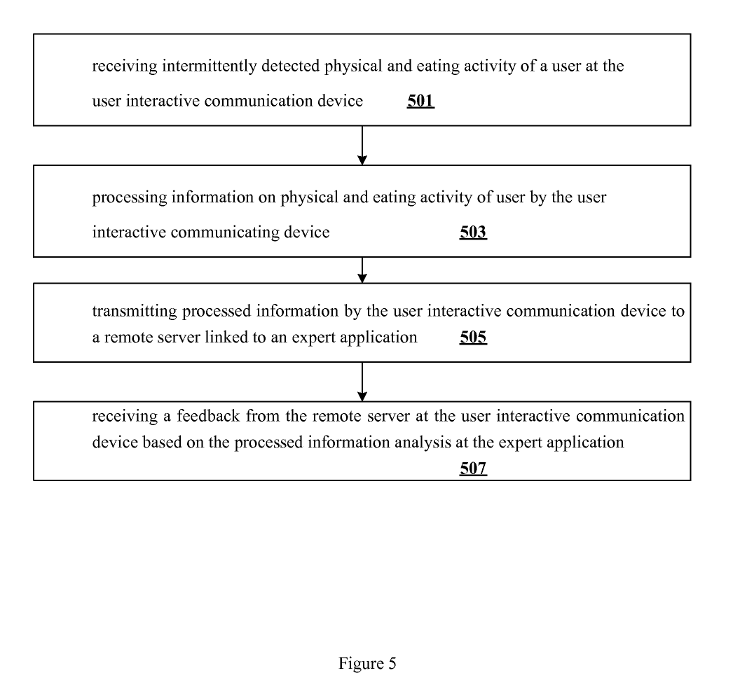

FIG. 5 represents a method of obesity management through remote monitoring and feedback as per one embodiment of present invention. The method provides accurate and timely measurements of parameters of the user related to their physical and eating activity. No user involvement may be required in providing measurements as the same may be intermittently detected.

The timely measurement of user parameters that effect obesity may be done by receiving 501 intermittently detected physical and eating activity of a user at the user interactive communication device. The user interactive communication device may be made available to the user, which may enable him monitor the information.

The method further involves processing 503 information on physical and eating activity of user by the user interactive communicating device. The information available at the user interactive communication device may be grouped or further processed based on the protocol of communication for further transmission.

The information of the user needs to be monitored for providing feedback to the user. The same is accomplished by transmitting 505 processed information by the user interactive communication device to a server. The server may be linked to an expert application. Various expert applications may be provided which may help in devising a medicine and training schedule to the user. Analysis of the processed information may be undertaken in the expert application in providing a feedback. Alternatively, an automated feedback action may be provided by virtual coach application present in the server in response to pre-decided set of situations.

The server may format feedback provided by the expert application to ensure better readability by the user. The feedback is made available to the user by receiving 507 the feedback from the server at the user interactive communication device. The feedback available to the user may reflect the latest detection of parameters in addition to the over-the-period data available at the server, analyzed in the expert application.

The present invention provides a method for continuous monitoring of the patient. Further, the system of the present invention is so programmed that intermittent detection of the parameters occurs without user involvement thereby providing timely and detailed information. Use of holistic system for obesity management further ensures uniformity in various measurements. Also, the user interactive sessions for calibration ensure a much accurate feedback by the expert applications.

While the present invention has been described with reference to certain exemplary embodiments, those skilled in the art will recognize that various modifications may be provided. Accordingly, the scope of the invention is to be limited only by the following claims.

What is claimed is:

1. An obesity management system, the system comprising:
a server;
a detecting device configured to intermittently measure various physical and eating activities of a user;
a user interactive communication device configured to intermittently receive information on the physical and eating activity from the detecting device;
wherein the user interactive communication device is configured to communicate with the server for sending information on physical and eating activity of the user, and receiving a feedback action.

2. The system of claim 1, wherein the information comprises physical movement of the user.

3. The system of claim 1, wherein the information comprises food intake detail of the user.

4. The system of claim 1, wherein the information comprises muscle contraction and respiration pattern detail of the user.

5. The system of claim 1, wherein the detecting device is implanted in the user.

6. The system of claim 1, wherein the detecting device is wearable by the user.

7. The system of claim 1, wherein the detecting device is self powered.

8. The system of claim 1, wherein the user interactive communication device is a personal digital assistance device.

9. The system of claim 1, wherein the user interactive communication device is a personal computing device.

10. The system of claim 1, wherein the user interactive communication device is a remotely programmable device configured to receive the feedback action inform of a script.

11. The system of claim 1, wherein the period of detecting information by the detecting device is variable.

12. The system of claim 1, wherein the feedback action is generated using an expert application linked with the server, in response to the information maintained at the server.

13. The system of claim 1, wherein an automated feedback action is generated using a virtual coach application available at the server.

14. The system of claim 12, wherein the expert application is a medical care provider application.

15. The system of claim 12, wherein the expert application is a dietician and physical trainer application.

16. The system of claim 12, wherein the expert application is a coaching program content provider application.

17. The system of claim 1, further comprising a neuro-stimulator configured to stimulate a feeling of satiety to eating.

18. An apparatus for physical and eating activity detection comprising:
a microprocessor;
a rechargeable power storage device;
a power source;
an accelerometer configured to detect physical movement of a user;
a diagnostic impedance meter configured to detect food intake details of the user;
a radio frequency transmitter configured to transmit information on physical movement of the user and food intake details of the user on instructions of the microprocessor.

19. The apparatus of claim 18, wherein the power source is a kinetic energy power generator to generate current for providing power to the apparatus.

20. The apparatus of claim 18, wherein the power source is a radio frequency power receiver generating current from radio frequency signals for providing power to the apparatus.

21. The apparatus of claim 18, further comprising:
a neuro-stimulator configured to stimulate a feeling of satiety to eating.

22. The apparatus of claim 18, further comprising:
a muscle contraction and respiration pattern sensor configured to detect and record muscle contraction and respiration pattern through measurement of electrical variation.

23. A method for remote obesity management, the method comprising the steps of:
receiving intermittently detected physical and eating activity of a user at the user interactive communication device;
processing information on physical and eating activity of user by the user interactive communicating device;
transmitting processed information by the user interactive communication device to a server linked to an expert application;
receiving a feedback from the server at the user interactive communication device based on the processed information analysis at the expert application.

24. The method as in claim 23, wherein the information comprises physical movement of the user.

25. The method as in claim 23, wherein the information comprises food intake of the user.

26. The method as in claim 25, wherein the food intake information is detected by measuring electrical variation around the stomach of the user.

27. The method as in claim 23, wherein the information comprises muscle contraction and respiration pattern information of the user.

28. The method as in claim 23, wherein the physical and eating activity of the user is detected using an implanted device.

29. The method as in claim 23, wherein the physical and eating activity of the user is detected using a wearable device

30. The method as in claim 23, wherein the user interactive communication device is a personal digital assistance device.

31. The method as in claim 23, wherein the user interactive communication device is a personal computing device.

32. The method as in claim 23, wherein the user interactive communication device is a remotely programmable device configured to receive the feedback action inform of a script.

33. The method as in claim 23, wherein the period of detecting information by the detecting device is variable.

34. The method as in claim 23, wherein the expert application is a medical care provider application.

35. The method as in claim 23 wherein the expert application is a dietician and physical trainer application.

36. The method as in claim 23, wherein the expert application is a coaching program content provider application.

37. The method as in claim 23, wherein an automated feedback action may be provided by a virtual coach application available at the server.
